(19) United States
(12) Patent Application Publication
(10) Pub. No.: US 2012/0197173 A1
(43) Pub. Date: Aug. 2, 2012

(54) WOUND DRESSING PACKAGE AND APPLICATOR

(76) Inventor: Jeff Babitz, Carmel, NY (US)

(21) Appl. No.: 13/358,092

(22) Filed: Jan. 25, 2012

Related U.S. Application Data
(60) Provisional application No. 61/436,753, filed on Jan. 27, 2011.

(51) Int. Cl. A61F 13/02; A61F 15/00

(52) U.S. Cl. 602/54; 206/441

(57) ABSTRACT

A wound dressing package encloses an adhesive dressing, which has a contact surface and an opposed shielding surface. The contact surface of the adhesive dressing includes a treatment surface and an adhesive surface. The package includes a flexible carrier, which has an inner surface that faces the shielding surface of the adhesive dressing; and a protective cover, which has an inward surface that faces the contact surface of the adhesive dressing. The protective cover is attached to the flexible carrier to enclose the adhesive dressing. The protective cover is stiffer than the flexible carrier, and is configured to split apart adjacent the treatment surface when the wound dressing package is bent with the protective cover in tension.

### CROSS REFERENCE TO RELATED APPLICATIONS

The present application claims priority to U.S. Prov. App. 61/436,753 filed Jan. 27, 2011, and hereby incorporates herein by reference the disclosures thereof.

### FIELD OF THE INVENTION

The present invention relates to articles for medical treatment and, more particularly, to wound dressings.

### BACKGROUND OF THE INVENTION

Wound dressings are applied to wounds and scrapes on areas of the body that come into contact with dirt or bacteria, in order to stop bleeding, promote healing, and prevent an infection from developing. Wound dressings often are removed from their packages, and applied, in unsterile conditions. Even thoroughly washed hands, or sterile packaged gloves, can pick up a certain amount of bacteria in such conditions. Thus, bacteria can be inadvertently transferred to a wound during application of a wound dressing to the wound.

Therefore, there exists a need for an improved wound dressing that reduces the known risk of contaminating a wound during application of the dressing to the wound.

### SUMMARY OF THE INVENTION

In one embodiment of the present invention, a wound dressing package includes an adhesive dressing, a flexible carrier, and a protective cover. The adhesive dressing has a contact surface and an opposed shielding surface. The contact surface includes, at least at a middle portion, a treatment surface. The flexible carrier is adjacent the shielding surface of the adhesive dressing. The protective cover is adjacent the contact surface of the adhesive dressing. The protective cover is attached to the flexible carrier to enclose the adhesive dressing, and is stiffer than the flexible carrier, and is configured to split apart when the wound dressing package is bent with the protective cover in tension.

The adhesive dressing also includes an adhesive surface, which may overlap the treatment surface of the adhesive dressing.

The protective cover can be configured to split apart adjacent the treatment surface of the adhesive dressing.

The flexible carrier may include a peripheral region that extends beyond the edges of the adhesive dressing and is attached to the protective cover. The peripheral region of the flexible carrier may be folded over the edges of the adhesive dressing and attached to an outward surface of the protective cover. The peripheral region of the flexible carrier may be peelably adhered to the protective cover. The inner surface of the flexible carrier may be peelably adhered to the shielding surface of the adhesive dressing.

The protective cover may be non-adhesive to the contact surface of the adhesive dressing.

The protective cover may include a first member and a second member, each member having a proximal edge attached to the proximal edge of the other member at the middle portion of the adhesive dressing, and each member having lateral edges that extend away from the other member to a distal edge, with the attachment of the proximal edges being separable under bending strain of the protective cover.

The protective cover may be configured to curl away from the middle portion of the adhesive dressing upon splitting apart. The protective cover may be configured to curl by pre-tensioning an outward surface. The protective cover may be configured to curl by scoring an inward surface. In certain embodiments, the inward surface of the protective cover may be scored only partway across.

The protective cover may be configured to split apart by pre-scoring an outward surface of the protective cover. The protective cover may be configured to split apart by chemically weakening a portion of the protective cover.

According to another embodiment of the present invention, a wound dressing package includes an adhesive dressing that has a contact surface and an opposed shielding surface. The contact surface includes, at least at a middle portion, a treatment surface. The wound dressing package also includes a flexible carrier adjacent the shielding surface of the adhesive dressing; and a protective cover adjacent the contact surface of the adhesive dressing. The protective cover is attached to the flexible carrier to enclose the adhesive dressing. The protective cover is stiffer than the flexible carrier, and is configured to split apart, adjacent the treatment surface of the adhesive dressing, when the wound dressing package is bent with the protective cover in tension.

In an aspect of the present invention, sterile application of a wound dressing is achieved by packaging the wound dressing between a flexible carrier and a protective cover, which is configured to split apart under bending tension. The protective cover is split open by bending the flexible carrier, wound dressing, and protective cover with the protective cover in tension toward a wound. Subsequent to splitting of the protective cover, a treatment surface of the wound dressing is pressed against the wound, without direct handling of the wound dressing.

These and other objects, features and advantages of the present invention will become apparent in light of the detailed description of the best mode embodiment thereof, as illustrated in the accompanying drawings.

### BRIEF DESCRIPTION OF THE DRAWINGS

FIG. 1 shows in exploded view a sterile wound dressing package according to one embodiment of the present invention;

FIG. 2 shows a first embodiment of a protective cover usable in a sterile wound dressing package;

FIG. 3 shows a second embodiment of a protective cover usable in a sterile wound dressing package;

FIG. 4 shows a third embodiment of a protective cover usable in a sterile wound dressing package; and

FIGS. 5A-5C show steps of a method of applying a sterile wound dressing according to an embodiment of the present invention.

### DESCRIPTION OF PREFERRED EMBODIMENTS

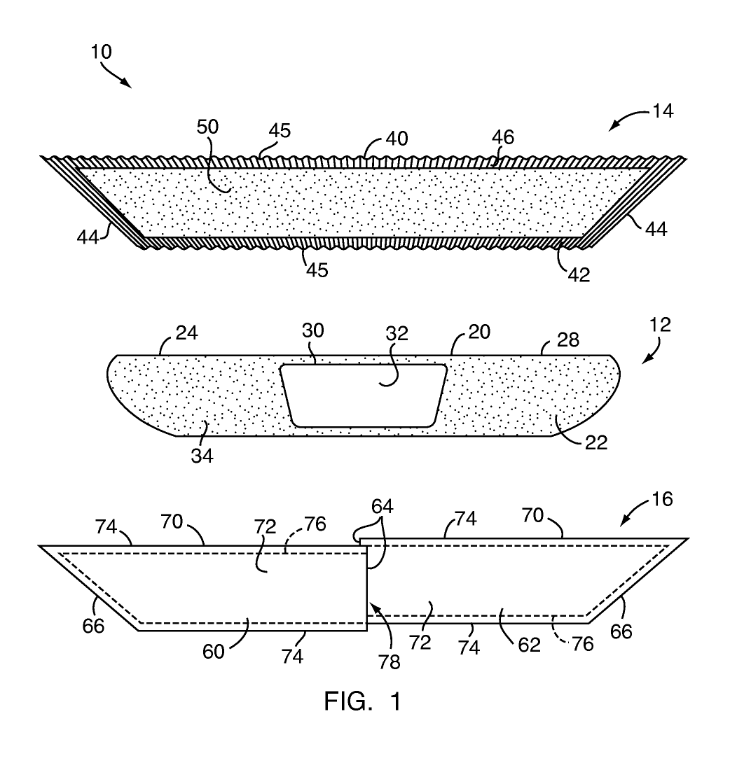

Referring to FIG. 1, in one embodiment of the present invention, a sterile wound dressing package 10 includes an adhesive dressing 12 fully enclosed between a flexible carrier 14 and a protective cover 16, which together define the sterile package 10.

The adhesive dressing 12 includes an upper or shielding surface 20, a lower or contact surface 22, and a perimeter edge 24. The upper surface 20 of the adhesive dressing 12 is formed by a non-adhesive upper surface of a tape or elongated flexible substrate 28. The tape 28 supports, at a middle portion of its lower surface 22, a treatment pad 30 that defines a treatment surface 32 of the adhesive dressing 12. At least on portions outlying or distal from the treatment pad 30, the lower surface of the tape 28 is provided with a skin-safe peelable adhesive. For example, Dow Corning product lines 7-9700, 7-9800, 7-9850, or 7-9900 are non-limiting examples of “skin-safe peelable adhesives” as understood by the skilled worker. The skin-safe peelable adhesive regions define an adhesive surface 34.

In certain embodiments, the treatment pad 30 can be any of a compress, transdermal patch, gauze, felt, or wad of fiber, which may be impregnated with topical pharmaceuticals. Other variants of treatment pads will be appreciated by those skilled in the art. In some embodiments, the tape lower surface 22 may incorporate the treatment surface 32 as well as the adhesive surface 34. In certain embodiments, at least a portion of the treatment pad 30 can incorporate a skin-safe peelable adhesive, such that the treatment surface 32 can overlap the adhesive surface 34.

In some embodiments, at least one of the tape 28, or the treatment pad 30, may be “breathable” or permeable to air and water vapor, while still providing a barrier to microbes or liquids. Other variations of the adhesive dressing 12 will be appreciated by those skilled in the art.

As shown in FIG. 1, the flexible carrier 14 is substantially flat or planar and includes an upper or outer surface 40, a lower or inner surface 42, opposed distal edges 44, lateral edges 45 connecting the ends of the distal edges, and a peripheral region 46 that includes the distal and lateral edges. The peripheral region 46 of the flexible carrier 14 extends beyond the perimeter edge 24 of the adhesive dressing 12. The inner surface 42 of the flexible carrier 14 adheres to the upper surface 20 of the adhesive dressing 12 by a layer of low tack adhesive 50. The low tack adhesive 50 holds the adhesive dressing 12 in place so that the flexible carrier can be used for application of the adhesive dressing 12 to the wound. The low tack adhesive 50 may extend beyond the perimeter edge 24 of the adhesive dressing 12, across at least a portion of the flexible carrier's peripheral region 46.

The flexible carrier 14 and the protective cover 16 may be fabricated of paper, plastic, or any flexible material. In some embodiments, the protective cover 16 may be fabricated from material that is stiffer than the flexible carrier 14. Both the flexible carrier 14, and the protective cover 16, provide barriers to infiltration of microbes or other contaminants.

As shown in FIG. 1, the protective cover 16 is attached to the flexible carrier 14 to fully enclose the adhesive dressing 12 in the package 10, such that sterile conditions can be maintained around the adhesive dressing. In the embodiment shown in FIG. 1, the protective cover 16 includes a first member 60 and a second member 62. Each of the first and second members includes a proximal edge 64, a distal edge 66, an upper or inward surface 70, a lower or outward surface 72, and lateral edges 74 that extend from each end of the proximal edge to a corresponding end of the distal edge.

The upper surface 70 of the protective cover 16 is not adhesive to the adhesive portions of the treatment surface of the adhesive dressing 12. Thus the protective cover 16 can easily separate from the wound dressing 12 at the time of application.

Adjacent to its distal and lateral edges 66, 74, the protective cover 16 includes a perimeter portion 76 (bounded by broken lines in FIG. 1). The perimeter portion 76 is attached to the peripheral region 46 of the flexible carrier 14 to form the package 10, which encloses the adhesive dressing 12 to keep the adhesive dressing sterile. For example, as shown in FIG. 1, the low tack adhesive layer 50 adheres the peripheral region 46 to the perimeter portion 76.

It will be appreciated that the peripheral region of the flexible carrier 14 may be attached to the protective cover 16 by adhesion, crimping, cohesion, thermal fusion, folding over, or any other available means for attachment. Notably, the chosen material or structure for attachment will permit the peripheral region 46 of the flexible carrier 14 to substantially disengage from the protective cover 16 during application of the adhesive dressing 12, such that the package 10 can be removed from the adhesive dressing during application.

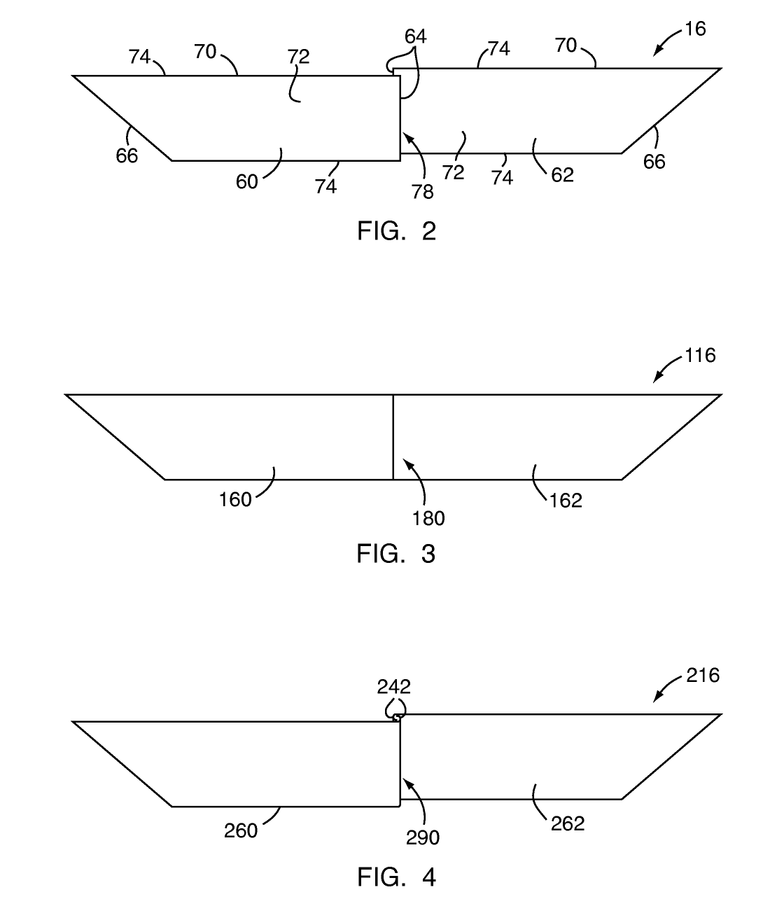

As mentioned, the protective cover 16 is configured to separate from the flexible carrier 14 during application of the wound dressing 12 to a wound. In particular the protective cover 16 is configured to split apart, adjacent the treatment surface 32, during application of the adhesive dressing 12. In an embodiment as shown in FIGS. 1-2, the proximal edges 64 of the first and second members 60, 62 join in an overlap relationship 78 in registry with the pad 30 of the adhesive dressing 12. The proximal edges 64 are joined together by adhesion, by cohesion, by folding or crimping, by thermal fusion, or by any other available means for attachment. The attachment mode of the proximal edges 64 is chosen such that the overlap 78 will split apart when the package 10 is subjected to suitable bending strain with the protective cover 16 in tension. In some embodiments, the edges 64 will separate when the protective cover 16 is bent from its normally planar configuration by about one quarter (0.25) inch downward displacement of the treatment pad 30. In select embodiments, the edges 64 will separate as the protective cover 16 is bent to no more than about one half (0.5) inch downward displacement.

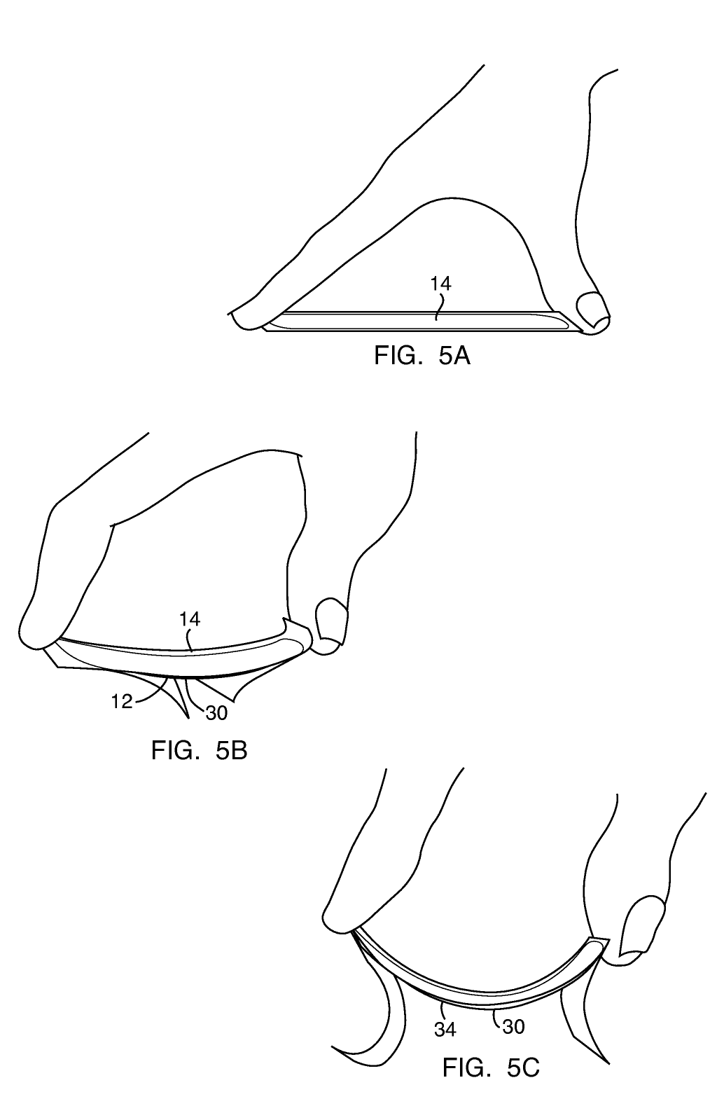

Desirably, the edges 64 will separate when the protective package 10 is bent by the fingers of one hand. Desirably, the lower surface 72 of the protective cover 16 is configured such that the first and second members 60, 62 will curve or curl away from the treatment pad 30 on separation of the overlap relationship 78, as shown for example in FIG. 5 (further discussed below). For example, the protective cover 16 may be constructed as a bilayer with the lower or outward layer 72 of each member pretensioned relative to the upper or inward layer. Alternatively, the inward surface 70 may be scored or otherwise relaxed relative to the outward surface 72. Other variations will be apparent to those of ordinary skill, in light of the present disclosure.

Referring to FIG. 3, according to another embodiment of the present invention, a protective cover 116 includes a first member 160 and a second member 162 that are defined by a breakable score 180 running across a middle portion of the protective cover 116. When the protective cover 116 is attached to the flexible carrier 14 to form a package enclosing an adhesive dressing 12, the breakable score 180 is disposed in registry with the pad 30 of the adhesive dressing 12. The breakable score 180 is weakened so as to rupture or split apart when subjected to suitable bending strain. Desirably, the protective cover 116 is scored or is pre-tensioned or is otherwise configured such that the first and second members 160, 162 will curve or curl away from the treatment pad 30 on separation of the breakable score 180.

Referring to FIG. 4, according to yet another embodiment of the present invention, the protective cover 216 is provided with a center joint 290. When the protective cover 216 is attached to the flexible carrier 14 to form a package enclosing an adhesive dressing 12, the center joint 290 is disposed in registry with the pad 30 of the adhesive dressing 12. The center joint 290 comprises a hook portion 292 of the first member 260 and a hook portion 292 of the second member 260. The hook portions of the first and second member are adapted to disengage at the time of application. In some embodiments, the hook portions 292 will disengage when the protective cover 216 is bent from its normally planar configuration.

In operation, as shown in FIG. 5, a sterile wound dressing package is placed above but not contacting a wound on the skin. With one hand, the opposed distal edges of the flexible carrier 14 are bent towards each other. The first and second members of the protective cover break apart, thereby opening the sterile package to expose the adhesive dressing 12. Preferably, on breaking apart, each of the first and second members of the protective cover curls or curves away from the treatment pad 30, thereby exposing the treatment pad for ease of application to the wound.

As pressure is applied downward onto the wound through the flexible carrier 14 and the adhesive dressing 12, the lower surface of the treatment pad 30 is applied directly to the wound without fingers coming into contact with the adhesive dressing 12. By further bending of the flexible carrier 14, combined with wiping motions outward from the treatment pad 30 toward distal edges 44 of the flexible carrier 14, the first and second members of the protective cover break free and may be discarded. The adhesive surface 34 adheres the dressing 12 to the healthy skin surrounding the wound, while the low tack adhesive layer 50 permits the flexible carrier 14 to be peeled from the wound dressing upper surface 20.

A primary advantage of the present invention is that peripheral region 46 of the flexible carrier remains joined to the perimeter portion 76 of the protective cover until application of the adhesive dressing 12. Thus, the adhesive dressing contact surface 22 does not contact anything except for the wound and surrounding skin. Thus, it is possible for the treatment surface 32 to remain sterile at all times until contact with the wound. By providing for application of the adhesive dressing 12 without direct handling, the package 10 substantially reduces any risks of contaminating the adhesive dressing or the wound. Pre-cleaning of the wound and surrounding skin, for example by sterile or antiseptic wash or swab, can further reduce risk of wound contamination.

Another advantage of the present invention is the efficiency with which the adhesive dressing 12 can be applied. More specifically, the present invention permits application of the adhesive dressing 12 by simply bending together the distal edges of the flexible carrier 14, using only the fingers of a single hand. Bending the flexible carrier 14 in the direction of the wound, with a single hand, forces the first and second members of the protective cover 16 to separate from the middle portion of the adhesive dressing 12, while further bending will completely disengage the protective cover from the adhesive dressing 12. Thus, speedy single-handed application reduces the duration of wound exposure. The single-handed application also reduces or eliminates risk of skin-borne contaminants (such as MRSA or multidrug-resistant staph aureus) reaching the wound.

Yet another advantage of the present invention is the flexibility of design. Notably, the flexible carrier 14 and protective cover 16 can be fabricated as any desired size to accommodate any type of adhesive dressing 12, including large cold- or hot-compresses.

A further advantage of the present invention is the economy of the design. Both the flexible carrier 14 and the protective cover 16 are fabricated from readily available and inexpensive materials, such as plastic or paper.

Although this invention has been shown and described with respect to the detailed embodiments thereof, it will be understood by those skilled in the art that various changes in form and detail thereof may be made without departing from the spirit and the scope of the invention.

What is claimed is:

1. A wound dressing package comprising:
an adhesive dressing;
a flexible carrier; and
a protective cover attached to the flexible carrier to enclose the adhesive dressing,
wherein the protective cover is stiffer than the flexible carrier, and is configured to split apart when the wound dressing package is bent with the protective cover in tension.

2. The wound dressing package as claimed in claim 1, wherein the protective cover is configured to split apart adjacent a treatment surface disposed at a middle portion of the adhesive dressing.

3. The wound dressing package as claimed in claim 1, wherein the adhesive dressing (12) has a contact surface (22) and an opposed shielding surface (20), said contact surface including, at least at a middle portion, a treatment surface (32); wherein the flexible carrier (14) is disposed adjacent the shielding surface of the adhesive dressing; and wherein the protective cover (16) is disposed adjacent the contact surface of the adhesive dressing.

4. The wound dressing package as claimed in claim 3, wherein the contact surface of the adhesive dressing includes, at least at a portion outlying the treatment surface, an adhesive surface (34).

5. The wound dressing package as claimed in claim 4, wherein the adhesive surface of the adhesive dressing overlaps the treatment surface of the adhesive dressing.

6. The wound dressing package as claimed in claim 1, wherein the flexible carrier includes a peripheral region (46) that extends beyond the adhesive dressing and is attached to the protective cover.

7. The wound dressing package as claimed in claim 1, wherein the flexible carrier is peelably adhered to the adhesive dressing.

8. The wound dressing package as claimed in claim 1, wherein the protective cover is non-adhesive to the adhesive dressing.

9. The wound dressing package as claimed in claim 1, wherein the protective cover includes a first member (60) and a second member (62), each member having a proximal edge (64) attached to the proximal edge of the other member at the middle portion of the adhesive dressing, and each member having lateral edges that extend away from the other member to a distal edge (66), and the attachment of the proximal edges is separable under bending strain of the protective cover.

10. The wound dressing package as claimed in claim 1, wherein the protective cover is further configured to curl away from the adhesive dressing upon splitting apart.

11. The wound dressing package as claimed in claim 10, wherein the protective cover is configured to curl by pre-tensioning an outward surface (72) of the protective cover relative to the inward surface of the protective cover.

12. The wound dressing package as claimed in claim 10, wherein the protective cover is configured to curl by scoring the inward surface of the protective cover.

13. The wound dressing package as claimed in claim 12, wherein the inward surface of the protective cover is scored only partway across.

14. The wound dressing package as claimed in claim 1, wherein the protective cover is configured to split apart by pre-scoring an outward surface of the protective cover.

15. The wound dressing package as claimed in claim 1, wherein the protective cover is configured to split apart by chemically weakening a portion of the protective cover.

16. A wound dressing package comprising:
an adhesive dressing (12) having a contact surface (22) and an opposed shielding surface (20), said contact surface including, at least at a middle portion, a treatment surface (32);
a flexible carrier (14) adjacent the shielding surface of the adhesive dressing; and
a protective cover (16) adjacent the contact surface of the adhesive dressing, said protective cover being attached to the flexible carrier to enclose the adhesive dressing,
wherein the protective cover is stiffer than the flexible carrier, and is configured to split apart, adjacent the treatment surface of the adhesive dressing, when the wound dressing package is bent with the protective cover in tension.

17. The wound dressing package as claimed in claim 16, wherein the protective cover is configured to curl away from the treatment surface of the adhesive dressing upon splitting apart.

18. The wound dressing package as claimed in claim 17, wherein the flexible carrier is peelably adhered to the adhesive dressing and to the protective cover, and the protective cover is not adhered to the adhesive dressing.

19. A method for sterile treatment of a wound, comprising:
bending a wound dressing package of a flexible carrier, an adhesive dressing, and a protective cover to split the protective cover toward a wound, thereby exposing a treatment surface of the adhesive dressing; and
pressing the treatment surface against the wound, without directly handling the adhesive dressing.

20. The method as claimed in claim 19, further comprising: after pressing the treatment surface against the wound, removing the flexible carrier and the protective cover from the adhesive dressing, without directly handling the adhesive dressing.
